(19) United States
(12) Patent Application Publication
(10) Pub. No.: US 2014/0302163 A1
(43) Pub. Date: Oct. 9, 2014

(54) Water With Improved Transdermal and Cellular Delivery Properties and Methods Of Manufacture And Use Thereof

(71) Applicant: Subtech Industries, LLC, Stuart, FL (US)

(72) Inventors: David Sanchez, Port Saint Lucie, FL (US); Len Deweerdt, Port Saint Lucie, FL (US)

(73) Assignee: Subtech Industries, LLC, Stuart, FL (US)

(21) Appl. No.: 14/214,945

(22) Filed: Mar. 16, 2014

Related U.S. Application Data
(60) Provisional application No. 61/788,206, filed on Mar. 15, 2013.

(51) Int. Cl. A23L 1/29; C02F 1/30; A61K 33/00; A61K 36/02; A61K 31/714; A61K 31/519; A61K 31/593; A23L 2/52; A61K 31/194

(52) U.S. Cl. CPC A23L 1/293; A23L 2/52; C02F 1/30; A61K 33/00; A61K 31/194; A61K 31/714; A61K 31/519; A61K 31/593; A61K 36/02
USPC 424/600; 426/2; 426/66; 210/251; 210/198.1; 210/201; 210/202; 514/569; 514/52; 424/195.17; 514/789

(57) ABSTRACT

A process for preparing conventional water sources for providing “ideal state” cellular wellness benefits via topical hydration and natural transdermal, epithelial or epicellular absorption. The invention's process considers preconditioned water sources that have removed solids, dissolved solids, contaminants—both organic and inorganic and subsequently the method purifies the water respective of removing pathogens, and structures the water for cellular uptake in the form of vicinal grade water, with improved characteristics of ideal frequency, cluster size, pH, before safely introducing negative ORP (highly ionized state) to the water structure and organic nutrients or other supplemental pharmaceutical grade organic materials onto the skin or epithelial/epicellular environments surrounding tissue and cells.

### INCORPORATION BY REFERENCE AND CLAIM OF PRIORITY

This application claims priority to U.S. provisional patent application No. 61/788,206 filed on Mar. 15, 2013, the contents of which are expressly incorporated by reference. The contents of all references cited herein are expressly incorporated in their entirety.

### BACKGROUND OF THE INVENTION

Water is ubiquitous in biology and plays roles which are well known and many which we do not fully appreciate. Water plays a primary role in biology for solubilizing and transporting molecules required to maintain life. The art primarily teaches ingestion of water solubilized molecules to meet nutritional or therapeutic needs.

Water is universally recognized as a good solvent for charged and polar solvents and a poor solvent for hydrocarbons.

The art has recognized that intracellular water, water located within cells, appears to be different than water as we generally know it in normal bulk phase. Phillipa Wiggins, Role of Water in Some Biological Processes, Microbiological Reviews. Vol 54 (4) p432-449 (1990). In fact, there appears to be 3 forms of water, water which is bound to surfaces, vicinal water associate with surfaces but not bound to them and ordinary aqueous water. Each of these three forms has different properties.

In recent years the literature has given greater attention to vicinal water and its role in cellular function.

Many enzymatic reactions are oxidation-reduction reactions in which one compound is oxidized and another compound is reduced. The ability of an organism to carry out oxidation-reduction reactions depends on the oxidation-reduction state of the environment, or its reduction potential.

In aqueous solutions, the reduction potential is a measure of the tendency of the solution to either gain or lose electrons when it is subject to change by introduction of a new species. A solution with a higher (more positive) reduction potential than the new species will have a tendency to gain electrons from the new species (i.e. to be reduced by oxidizing the new species) and a solution with a lower (more negative) reduction potential will have a tendency to lose electrons to the new species (i.e. to be oxidized by reducing the new species). Oxidation Reduction Potential (“ORP”) is generally measured by means of a electrode. Redox meters are well known in the art.

However, internal delivery of molecules is not always efficient or even effective due to the many mechanisms the body has to prevent contamination including but not limited to filtering by liver and kidneys and active pumps present in the gut such as p glycoprotein.

The art has tried to create ways to improve the properties of water for various biological purposes. Various attempts have been made to produce structured water through electrochemical cells or filtration media.

Below are representative patents and published patent applications relevant to structured water.

JP 20044285036 for Skin Conditioning Agent to Hiroshima Kasei. This application teaches a skin conditioning agent which is produced electrolytically containing a reduced water having a neutral pH and a negative 400 redox.

JP 2006096667 and U.S. Pat. No. 5,776,346 teach that water produced by ion exchange resins including tourmaline and aluminum oxide having negative oxidation reduction potential is ingestible to lower blood sugar.

JP2006326374 teaches the use of electrodes to modify the redox potential of water along with use of tourmaline.

JP2003335655A2 discloses a mineral water generated by passing natural water through a column that includes tourmaline. No values appear to be given for redox.

US 20040118775 A1 teaches the use of bubbling hydrogen gas through water to produce a product with a ph from 6.5-9 and redox values from −150 to −900.

U.S. Pat. No. 5,880,048 A for Granular Ceramic for Water Deoxidation and method of Producing the same Sato; Kazuo et al.

KR100647036B1 for Filter for Generating Alkaline Reduced Water and Apparatus Using the Same to Jeon Hyoung Tag. This patent discloses the use of ORP balls but does not appear to reach the negative redox levels you achieve. (no English language version available)

US20120213756A1 teaches the use of oral fulvic acid to scavenge free radicals and transport nutrients. It does not teach transdermal use.

US2006/7067155 Anti-inflammatory humate compositions and methods of use thereof teaches the use of humates and fulvics as supplementation that can reduce inflammation.

Phytochemicals and micronutrients sourced from plants are now known to impact gene expression in animals according to recent studies. Cell Research 22:3-5, doi:1038/cr.2011.164; published online October 2011 article entitled “Ingested plant miRNAs regulate gene expression in animals” clearly showed phenotypical changes related to proteomic influences in animals tested.

US2010/0092399 A1 for Methods of Treating or Preventing Inflammation and Hypersensitivity with Oxidative Reduction Potential Water Solution to Hojabr Alimi. This published patent application teaches the use of high positive ORP water generated electrolytically.

US2010/0316776 for Compositions and Methods for Producing Stable Negative ORP in Consumable Materials to Dusan Miljkovic. This published patent application teaches using reducing compounds to generate high negative ORP water. KHCO3 is primarily used and ascorbic acid used to adjust pH.

US 2006/0275498 for Processed Water and Therapeutic Uses Thereof to David Bagley discloses the use of electrolytically obtained negative ORP water to treat conditions.

U.S. Pat. Nos. 5,711,950 and 6,033,678 teach the preparation of microclustered water using steam, magnetic fields and far infra-red to ultraviolet spectrum range light.

U.S. Pat. No. 7,291,314 for Activated water apparatus and methods teaches a structured water having an ORP below −350 and a cluster size below 4 and a pH below 4 or higher than 10 produced via RD plasma.

In a recent study, fungal activity was altered by water borne frequency; refer to patent WO2007068831 (A2)—2007 Jun. 21, Luc Montagnier, METHOD FOR CHARACTERISING A BIOLOGICALLY ACTIVE BIOCHEMICAL ELEMENT BY ANALYSING LOW FREQUENCY ELECTROMAGNETIC SIGNALS Electromagnetic field frequency memory in water as revealed by germination responses of fungal spores O. E. Ehinlafaa*, G. A. Ibitolab and O. Okunyec a Department of Physics, University of Ilorin, Ilorin, Nigeria

It is recognized that providing ionized water orally encourages free radical (toxin) removal both extra cellularly and intra-cellularly. Ionized water provided at the cellular level can increase negative cellular voltage via free hydrogen ions, expressed as negative ORP, which can provide additional bio-energy for cells directly, eliminate free radicals and encourage improve metabolic processes in the presence of available nutrients.

Research information shown in the paper “Magnesium Activated Hydrogen Ions and Biological Activity Empirical Analyses and Clinical Significance”, Cory J. Stephanson, Ph.D., Phi Sciences, Santa Cruz, Calif. and G. Patrick Flanagan, M. D., Ph.D., Phi Sciences, Cottonwood, Ariz., proved a scientific foundation for the understanding of the mechanism and associative use between magnesium active hydrogen ions and the parameters of oxidation reduction potential (ORP), biochemical involvement in energy producing intracellular reduction systems and cellular hydration.

Research performed by the 2nd Internal Medicine Dept. of Shiga Medical University and Dept. of Gastroenterology, National Ohkura Hospital, Japan tested alkaline water's ability to act as an antacid by measuring the pH of the stomachs of 6 volunteers before and after drinking alkaline water. See, http://www.lifeionizers.com/blog/news-updates/health-concerns-and-ailments/alkaline-water-health/#ixzz2BNKFUJKK. These tests showed that while stomach pH increased between 0.5 and 1.0, it did indicate that the acid reduction did eliminate many ions before any digestive uptake occurred. For that reason the primary value of oral use of alkaline structured water was in treating digestive issues versus improving internal conditions such as acidosis or interstitial fluid free radicals.

The inability to provide high negative ORP water orally without loss of its structured properties limits the potential to achieve the ORP levels that would provide benefit.

Deliberate administration of nutrients via topical means using just water as the transfer medium is unknown in the art.

It is unknown to use water alone as a means of reducing free radicals

Studies performed to look at presence of organic carcinogenetic solvents have shown that such solvents in water can be absorbed through the skin. For example, in studies performed by Chapel Hill North Carolina University, researchers observed that Trihalomethane contaminants in water were absorbed into the body via the skin during the normal process of showering. Comparison of Trihalomethanes in Tap Water and Blood; Miles, Singer et al Environ Sci Technol. 2002 Apr. 15; 36(8):1692-8.

Efforts to produce high ORP water usually involve the use of electrochemical cells which produce high levels of reactive compounds as a byproduct. These undesirable compounds frequently comprise halogens such as chlorine in various forms including, for example, chlorine gas, chloride ions, hydrochloric acid and/or hypochlorous acid, or one or more precursors thereof.

Equipment to generate high ORP water is generally electrolytic and has a high purchase price. Know known units can individually control ORP and pH to get a safe, vicinal grade water pH at the desired high negative ORP.

### BRIEF DESCRIPTION OF THE FIGURES

FIG. 1 is a flow chart of the process for making the present invention.

FIG. 2 is photograph of a darkfield microscopy image of a subjects red blood cells before treatment.

FIG. 3 is photograph of a darkfield microscopy image of a subject's red blood cells after treatment.

FIG. 4 is the Visual Contrast Sensitivity testing results of a subject before and after treatment with the water of the present invention.

### BRIEF DESCRIPTION OF THE INVENTION

The present invention relates to the use of water which is structured to provide smaller cluster sizes and having an extremely high negative oxidation reduction potential (“ORP”) while having a biologically acceptable pH. This effect is achieved by using water which has been purified by conventional means to at least one mega ohm of resistance. Such water is commonly produced for purposes of electronics manufacture and dialysis but suffers from having extremely high pH. The water is then processed by passing it through a resin to generate a high negative ORP. The resulting water can be optionally remineralized at this point before having the pH adjusted with a biologically acceptable acid, such as ascorbic acid. Inorganic and organic compounds intended for administration may be added and the resulting solution is administered to a living organism via either topical or oral routes of delivery. If desired, the frequency of the water can be modulated using frequency modulators known in the art.

Water of the present invention can be used alone to hydrate a living organism via topical application or through consumption orally. The water of the present invention reduces free radicals.

The Water of the present invention also serves as a novel means to deliver nutrients and pharmacologically active agents to the body.

The water of the present invention may be used to deliver active agents to the body via the skin.

Water of the present invention may be used to formulate cosmetics, dietary supplements, and pharmacologically active small molecules and peptides.

The sequence of the invention's components is a discretionary manufacturing decision and they are in no particular order with the exception of contaminant filtration and additive chambering. The components are as follow:

Intake coupling and shut off: The invention requires the ability to use a variety of pipe sizes and thread types and is therefore a regional design and uses shelf-available components. The water supply shut off should be fully restrictive and easy to use without stress to the piping or the user.

Piping between components: The invention requires piping that is rigid or semi-rigid and which can be readily coupled to components. Fittings and unions should be serviceable to allow component replacements.

Filtration: The invention uses filtration to remove insoluble contaminants. Removal of organics is recommended to avoid uptake of previously untreated ground or well water where levels are unknown. Manufacturing recommendations are by region and application. The filtration should be of such quality as to allow pure additives 100% solubility and be free of undesirable chemicals or other contaminants.

Pathogen removal: A clear glass or UV permeable PVC chamber or tube with unions must be designed and incorporated to allow a standard UV light to emit through the clear area so that pathogens are destroyed in the flow. Shelf available units may be obtained and bracket mounted to achieve the manufacturer's optimal focal and refractive characteristics when in use. A sensor to detect the light on condition should be visible to the user with a solenoid shut off included in the input side of the chamber or tube that can close the flow off until the UV unit is repaired. Alternately, UV may be applied directly to the surface area to be treated prior to the solution dispersal to the epithelial/epidermal tissue.

pH conditioning: Fixed magnets or electrical field inducement or addition of bicarbonates can be introduced to further treat the water. Effective alkaline ranges should normally not exceed those found as naturally occurring which are consumable for humans or animals.

Chambering for additives: The invention incorporates one or more mixing chambers for dry additives such that trans-dermal or epithelial soluble molecular compounds may be added according to recommended dosages, along with enhancers or drivers, where available. Dry material is applied in this invention to manage concentrations at the volume and flow for normal line pressures, normally not exceeding 80-90 PSI. An integrated injector that is removable is recommended to allow for improving efficacy and efficiency in the mixing process as well as conservation of additives, when applicable.

Proper flow for mixing is a function of shelf available components and variability by manufacturer should be compared to optimize a steady state of limited, known concentrated delivery to allow extended use without clogging. It is recommended that flow restrictors and metering valves be incorporated into the mixing chamber to assure further controlled concentration and consumption of the dry additives. Selection of soluble additives is also a critical element in each application.

In-line heating: The invention uses normally available heated water from external sources. Shelf available continuous flow heating may be incorporated at a point in the flow but due to size may require separate mounting brackets and attach before filtration.

Electrical: Recommended application of power for components is for fully enclosed, water tight connections and enclosures should be used following NEMA or comparable codes for power in wet environments. Low voltage DC is preferred where possible. Electrical component mounting and configuration should be considered for isolation and tied to ground fault interrupted circuits or as local, national or regional codes prescribe.

Solution Dispersal: The invention may use shelf available or custom dispersal devices such as showerheads, atomizers, nozzles, or other aqueous or fluid dispersal designs to achieve the desired 80% or greater organ area that the invention requires to deliver the broad uptake of the solution.

### DETAILED DESCRIPTION OF THE INVENTION

The goal of the present invention is to provide health benefits to an organism which is provided the structured water.

Vicinal water is water having the qualities of water found within living cells.

Cluster size refers to the number of molecules which are associated together.

An antioxidant's potential to supply electrons dispersed into a liquid can be tested by using an ORP (Oxidation/Reduction Potential) meter. Oxidized materials are shown as + above zero, antioxidants are either a low + or a negative reading. Lower numbers indicate more available electrons. For example, the antioxidant CoQ10 has an ORP of +49 mV; wheat grass juice has an ORP of −120 mV. The negative reading of wheat grass juice gives it a significantly higher potential for donating electrons and neutralizing free radicals than CoQ10.

Biologically acceptable pH is that pH which will not harm living cells. Biologically acceptable pH is generally considered to be between about 6 to about 8, and is preferably between about 7 to about 7.5 and most preferably about 7.35 to about 7.45.

Dietary supplements are non-food products delivered to a living thing with a goal of improving its nutrition. Dietary supplements can be vitamins and minerals, botanicals and animal derived products. Common examples include Acai, Aloe Vera, Anabolic Steroids, Astragalus, Bilberry, Bitter Orange, Black Cohosh, Botanical Dietary Supplements, Butterbur, Calcium, Carnitine, Cartilage, Cat's Claw, Chamomile, Chasteberry, Chondroitin, Chromium, Cinnamon, Coenzyme Q10, Colloidal Silver, Cranberry, Dandelion, Dietary Supplements, Echinacea, Ephedra, Essiac/Flor-Essence, European Elder, Evening Primrose Oil, Fenugreek, Feverfew, Fish Oil, Flaxseed, Folate, Frequently Asked Questions, Garlic, Ginger, Ginkgo, Ginseng, Glucosamine, Goldenseal, Grape Seed Extract, Green Tea, Hawthorn, Herbal Dietary Supplements, Hoodia, Horse Chestnut, Iodine, Iron, Kava, Lavender, Licorice Root, Magnesium, Melatonin, Milk Thistle, Mistletoe, Multivitamin/mineral Supplements, Noni, Omega-3 Fatty Acids, PC-SPES, Peppermint Oil, Red Clover, Sage, SAMe (S-Adenosyl-L-Methionine), Saw Palmetto, Selenium, Soy, St. John's Wort, Tea, Thunder God Vine, Turmeric, Valerian, Vitamin A, Vitamin B12, Vitamin B6, Vitamin C, Vitamin D, Vitamin E, Vitamin K, Yohimbe, Zinc.

Phytochemicals, including but not limited to the following: alkaloids, caffeine, theobromine, theophylline; anthocyanins, cyanidin, malvidin; carotenoids, beta-carotene, lutein, lycopene, coumestans, flavan-3-ols; flavonoids, epicatechin, hesperidin, isorhamnetin, kaempferol, myricetin, naringin, nobiletin, proanthocyanidins, quercetin, rutin, tangeretin, hydroxycinnamic acid, chicoric acid, coumarin, ferulic acid, scopoletin; isoflavones, daidzein, genistein; lignans, silymarin; monophenols, hydroxytyrosol; monoterpenes, geraniol, limonene; organosulfides, allicin, glutathione, indole-3-carbinol, isothiocyanates, sulforaphane; phytoplankton, other phytochemicals, damnacanthal, digoxin, phytic acid; phenolic acids, capsaicin, ellagic acid, gallic acid, rosmarinic acid, tannic acid; phytosterols, beta-sitosterol; saponins; stylbenes, pterostilbene, resveratrol; triterpenoid, ursolic acid; xanthophylls, astaxanthin and beta-cryptoxanthin.

Homeopathic agents are naturally occurring substances delivered in diluted form. Common homeopathic agents include Aconite Aconitum napellus (Monkshood), Aesculus Aesculus hippocastaneum (Horsechestnut), Agaricus, Allium cepa (Onion), Aloe, Ambra grisea (Amber), Anac. Anacardium, (the Marking Nut), Ant. Crud. Antimonium crudum, Ant. Tart. Antimonium tartaricum, Apis mellifica (Honey bee), Arnica montana (Leopard's bane), Arg. Nit. Argentum nitricum, Arsenicum A., Aurum metallicum (Gold leaf), Baptisia, Belladonna (Atropa belladonna, Deadly Nightshade), Bellis perennis (Daisy), Berberis vulgaris (not notable), Bryonia alba, Cactus grandiflorus, Calc. Carb. Calcarea carbonica, Calc. Fluor. Calcarea fluorica, Calc. Phos. Calcarea phosphoricum, Calc. Sulph. Calcarea sulphuricum, Calendula officinalis, Camphora, Cantharis (Spanish fly), Carbo Veg. Carbo vegetabilis, Caulophyllum (Blue Cohosh), Causticum, Chamomilla, China O. China officinalis, Cimicifuga racemosa (Actaea racemosa), Cina, Cocculus, Coffea C., Colocynth Colocynthis (Squirting cucumber), Conium M. maculatum, Cuprum M. Cuprum metallicum, Digitalis purpurea (Foxglove), Drosera rotundifolia, Dulcamara Atropa dulcamara (Woody nightshade), Duck liver, Echinacea, Eup. Per. Eupatorium perfoliatum Euphrasia officinalis (eyebright), Ferrum Phosphoricum, Gelsemium (Yellow jasmine), Glonoin (Nitroglycerine), Graphites, Hamamelis (Witch-hazel), Hepar Sulph. Hepar sulphuris calcareum, Hypericum perforatum (St. John's wort), Ignatia amara (St. Ignatius bean), Ipecac Ipecacuanha, Kali Bich. (Potassium dichromate), Kali Carb. (Potassium carbonate), Kali Mur. Potassium chloride, Kali Phos. (Potassium phosphate), Lac Can. Lac caninum (Dog's milk), Lachesis muta (Bushmaster snake), Ledum palustre (Marsh tea), Lycopodium, Magnesium Phosphoricum, Merc. Cor. Mercurius corrosivus, Merc Viv. Mercurius solubilis, Mezereum Daphne mezereum (Spurge olive), Nat. Mur. Natrum muriaticum, Nat. Phos. Natrum phosphoricum, Nat. Sulph. Natrum sulphuricum, Nitric acid, Nux Vomica, Opium, Petroleum Crude oil, Phos. Ac., Phosphorus, Phytolacca americana (Pokeweed), Platina, Lead, Podophyllum, Pulsatilla, Pyrogenium, Rhus Tox. (Poison ivy), Rumex crispus (Yellow dock), Ruta graveolens (Rue), Sabina Sabadilla Sanguinaria (Bloodroot) Sarsaparilla, Secale cornutum (Corn smut), Sepia officinalis (Cuttlefish), Silicea (Flint), Spigelia marilandica (Pinkroot), Sponge or Spongia tosta, Tin, Staphysagria, Stramonium Datura stramonium (Jimson weed, thornapple), Sulphur, Symphytum officinalis (Comfrey), Tarentula hispanica, Terbenthinum (Terpentine), Thuja occidentalis, Urtica urens (Stinging nettle), Valeriana officinalis, Veratrum Album, Zincum metallicum,

Pharmacologically active agents are small molecules and peptides delivered for the purpose of treating a medical condition. Examples of pharmaceologically active agents include: Analgesics include, e.g., para-aminophenol derivatives (e.g., acetaminophen), indole and indene acetic acids (e.g., etodalac), heteroaryl acetic acids (e.g., diclofenac and ketorolac), arylpropionic acids (e.g., ibuprofen), anthranilic acids (e.g., mefenamic acid and meclofenamic acid), enolic acids (e.g., tenoxicam and oxyphenthatrazone), nabumetone, gold compounds (e.g., gold sodium thiomalate), buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, methotrimeprazine, cinnamedrine hydrochloride, meprobamate, ketoprofen, flurbiprofen, naproxen, ramifenazone, meloxicam, fluazacort, celecoxib, rofecoxib, valdecoxib, nepafenac, ISV-205; angiogenesis inhibitors include, e.g., angiostatin (plasminogen fragment), vascular endothelial cell growth factor (VEGF), fibroblast growth factor (FGF), nitric oxide donors, antiangiogenic anithrombin III, cartilage-derived inhibitor (CD1), CD59 complement fragment, endostatin (collagen XVIII fragment), fibronectin fragment, gro-beta, heparinases, heparin hexasaccharide fragment, human chorionic gonadotropin (hCG), .alpha.-, .beta.-, and .gamma.-interferon, interferon inducible protein (IP-10), interleukin-12, kringle 5 (plasminogen fragment), metalloproteinase inhibitors (TIMPs), 2-methoxyestradiol, placental ribonuclease inhibitor, plasminogen activator inhibitor, platelet factor-4 (PF-4), prolactin 16 kD fragment, proliferin-related protein (PRP), retinoids, tetrahydrocortisol-S, thrombospondin-1 (TSP-1), transforming growth factor-beta (TGF-b), vasculostatin, vasostatin (calreticulin fragment), apolipoprotein E, TBC-2576; antiasthmatics include, e.g., ketotifen and traxanox; antidepressants include, e.g., nefopam, oxypertine, amoxapine, trazodone, maprotiline, phenelzine, desipramine, nortriptyline, tranylcypromine, fluoxetine, doxepin, imipramine, imipramine pamoate, isocarboxazid, trimipramine, and protriptyline; antidiabetics include, e.g., biguanides (e.g., metformin), sulfonylurea derivatives (e.g., tolbutamide, chlorpropamide, acetohexamide, tolazamide, and glimepiride), .alpha.-glucosidase inhibitors (e.g., acarbose), thiazolidinediones (e.g., troglitazone), and metglinide analogs (e.g., repaglinide); antihypertensive agents include, e.g., propanolol, propafenone, oxyprenolol, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine; antineoplastics include, e.g., cladribine (2-chlorodeoxyadenosine), nitrogen mustards (e.g., cyclophosphamide, mechlorethamine, melphalan, and chlorambucil), ethylenimines and methylmelamines (e.g., hexamethylmelamine and thiotepa), alkyl sulfonates (e.g., busulfan), nitrosoureas (e.g., streptozocin, carmustine (BCNU), methyl-CCNU and analogs), trazenes (e.g., dacarbazinine (DTIC)), platinum coordination complexes (e.g., carboplatin and cisplatin), procarbazine, hydroxyurea, mitotane, aminoglutethimide, camptothecin phenesterine, paclitaxel, docetaxel, vinca alkaloids (e.g., vinblastine, vincristine, and vinorelbine), epidipodophyllotoxins (e.g., etoposide (VP-16) and teniposide), tamoxifen, and piposulfan; anxiolytics include, e.g., lorazepam, buspirone, prazepam, chlordiazepoxide, oxazepam, clorazepate dipotassium, hydroxyzine pamoate, hydroxyzine hydrochloride, alprazolam, droperidol, halazepam, chlormezanone, and dantrolene; enzyme inhibitors include, e.g., selegiline or its hydrochloride salt, lazabemide, rasagiline, moclobemide, entacapone, tolcapone, nitecapone, Ro 40-7592, clozapine, risperidone, olanzapine, and quetiapine; immunosuppressives include, e.g., calcineurin inhibitors (e.g., cyclosporine and tacrolimus (FK-506)), antiproliferative/antimetabolic agents (e.g., sirolimus, QP-2, taxol, actinomycin, dactinomycin, daunorubicin, angiopeptin, mitomycine, bleomycin, doxorubicin, epirubicin, mitomycin, idarubicin, anthracyclines, mitoxantrone, plicamycin, CMYC antisense, ABT-578, RestenASE, 2-chloro deoxyadenosine, PCNA ribozyme, rapamycin, folic acid analogs (e.g., methotrexate), fluorouracil (5-FU), floxuridine, cytarabine, mercaptopurine, thioguanine, pentostatin, cyclophosphamide, thalidomide, chorambucil, leflunomide, batimastat, and mizoribine), everolimus, azathioprine, cytoxan, mycophenolic acid, mycophenolate mofetil, and tranilast; antimigraine agents include, e.g., ergotamine, isometheptene mucate, and dichloralphenazone; sedatives and hypnotics include, e.g., barbiturates (e.g., pentobarbital and secobarbital), flurazepam hydrochloride, triazolam, and midazolam; calcium-channel blocker antianginals include, e.g., nifedipine and diltiazem; nitrate antianginals include, e.g., nitroglycerin, isosorbide dinitrate, pentaerythritol tetranitrate, and erythrityl tetranitrate; antipsychotics include, e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine; antimanics include, e.g., lithium carbonate; antiarrhythmics include, e.g., bretylium tosylate, esmolol, verapamil, amiodarone, encamide, digoxin, digitoxin, mexiletine, disopyramide phosphate, procainamide, quinidine sulfate, quinidine gluconate, quinidine polygalacturonate, flecamide acetate, tocamide, and lidocaine; antiarthritics include, e.g., phenylbutazone, sulindac, penicillanine, salsalate, piroxicam, indomethacin, meclofenamate, ketoprofen, auranofin, aurothioglucose, tolmetin, and tolmetin sodium; anti-gout agents include, e.g., colchicine and allopurinol; anticoagulants include e.g., danaparoid, lepirudin, dicumarol, acenocoumarol, heparin, heparin salts (e.g., heparin sodium), warfarin sodium, 4-hydroxycoumarin, phenprocoumon, indan-1,3 dione, anisindione, warfarin sodium, tissue factor pathway inhibitor (TFPI), tifacogin, ancrod, bromindione, clorindione, coumetarol, cyclocoumarol, 4-coumarinol, desirudin, dexran sodium sulfate, diphenadione, ethyl biscoumacetate, fluindione, hirudin, nadroparin calcium, nafamostat mesylate, oxazidione, phenindione, phosvitin, picotamide, sodium apolate, thrombocid, tioclomarol, warfarin, aprosulate sodium, ART 123, bivalirudin, BMS 189090, BMS 186282, BMS 189664, BMS 191032, corsevin M, CS 747, curdlan sulfate, DPC 423, DX 9065a, efegatran, fondaparinux sodium, GR 144053, inogatran, LB 30057, melagatran, MR 33, napsagatran, NSL 9403, SR 90107, YM 75466, ZK 805412, ZK 807834, OGS 15435, JTV 803, LY 287045, P 8720, RE 1492, Ro 43-8857, S 18326, S 31214, SK 549, SB 249417, SR 123781A, and UK 156406; thrombolytics/fibrinolytics include, e.g., urokinase, streptokinase, alteplase, phosphorylcholine, plasmin, plasminogen, angiokinase, anistreplase, prourokinase, reteplase, saruplase, tissue plasminogen activator, actinokinase, .alpha.2-antiplasmin, antithrombin, E 6010, fibrolase, lys-plasminogen, lanoteplase, lumbrokinase, metalloproteinase, monteplase, PAI proteinase inhibitor, pamiteplase, staphylokinase, and tenecteplase; antifibrinolytics include, e.g., aminocaproic acid; hemorheologic agents include, e.g., pentoxifylline; antiplatelet agents include, e.g., aspirin, ticlopidine, abciximab, clopidogrel, eptifibatide, tirofiban, and glycoprotein IIb/IIa inhibitors, argatroban, cilostazole, cloricromene, dalteparin, daltroban, defibrotide, dipyridamole, enoxaparin, iloprost, indobufen, isbogrel, lamifiban, lotrifiban nadroparin calcium, orbofiban, pamicogrel KBT 3022, plafibride, picotamide, ozagrel, ramatroban, reviparin sodium, ridogrel, roxifiban, satigrel, sibrafiban, sulotroban, taprostene, ticlopidine, triflusal, aminone, cilostamide, dialzep, enoximone, milrinone, naftazone, pimilprost, pimobendan, sarpogrelate, sulfinpyrazone, vapiprost, vesnarinone, xemilofiban, zaprinast, zeria Z 335, A 02131-1, camonagrel, cangrelor, DMP 728, DMP 802, elarofiban, EMD 122347 FK 633, FXV 673, ifetroban, L 734217, lefradafiban, MK 852, ON 579, R 99224, RGD 039, RGD 891, RPR 109891, Ro 48-3657, Ro 44-3888, S 1197, SDZ-GPI 562, SL 650472, SM 20302, SR 121566A, SR 121787A, TA 993, TAK 029, XV 454, XV 459, YC-1, aspalatone, BAY 41-2272, BM 531, BM 14515, C 186-65, CS 570, FR 158999, fradafiban, L 750034, linotroban, ME 3277, MED 27, NQ 12, NQ 301, NQ 304, NSL 9511, NSP 513, 4-pentynoic acid, 3-[[4-[[4-(aminomethyl)-phenyl]amino-]-1,4-dioxobutyl]-amino]-ethyl ester, RE 2047, SCH 79797, SM 10906, SR 25989, TP 9201, XJ 735, XR 300, XU 057, XU 063, XU 065, Y 909, ZD 2486, and ZD 9583; anti-apoptotics include, e.g., CGP 3466, CEP-1347/KT-7515, TCH-346, and WHI-P131; neurological agents include, e.g., timolol, dapiprazole, levobunolol, betaxolol, befunolol, carteolol, metipranolol, AMO-140, bunazosin, adaprolol, ISV-208, L-653328, cetamolol, H-216/44, KRG-332, levobetaxolol, metazosin, NCX-904, NCX-905, guanethidine, brimonidine, apraclonidine, AGN-195795, AGN-191103, AGN-190532, AGN-192172, AGN-193080, AGN-190837, talipexole, thiourea, dipivefrin, epinephrine, phenylephrine, cocaine, hydroxyamphetamine, naphazoline, tetrahydrozoline, levodopa, levodopa/carbidopa, levodopa/benserazide, amantadine, sumanirole, pergolide, pramipexole, ropinirole, bromocriptine, lisuride or 9,10 dihydrolisuride, apomorphine or N-propylnoraporphine, N-propyl noraporphine, PHNO, N-0437 (racemate) and N-9023 (purified negative enantiomer), cabergoline, ciladopa, ABT-431, lergotrile, DIB1508Y, and ABT418m; selective serotonin re-uptake inhibitors (SSRIs) include, e.g., paroxetine, and serataline; anticonvulsants include, e.g., valproic acid, divalproex sodium, phenyloin, phenyloin sodium, clonazepam, primidone, phenobarbitol, carbamazepine, amobarbital sodium, methsuximide, metharbital, mephobarbital, mephenyloin, phensuximide, paramethadione, ethotoin, phenacemide, secobarbitol sodium, clorazepate dipotassium, and trimethadione; anti-parkinsonian agents include, e.g., ethosuximide; antihistamines/antipruritics include, e.g., hydroxyzine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, and methdilazine; calcium regulators include, e.g., calcitonin and parathyroid hormone; antibacterials include, e.g., amikacin sulfate, aztreonam, chloramphenicol, chloramphenicol palirtate, clindamycin, clindamycin palmitate, clindamycin phosphate, metronidazole, gentamicin sulfate, lincomycin hydrochloride, tobramycin sulfate, vancomycin hydrochloride, polymyxin B sulfate, colistimethate sodium, and colistin sulfate; antibiotics include, e.g., neomycin, streptomycin, chloramphenicol, cephalosporin, ampicillin, penicillin, tetracycline, and ciprofloxacin; antifungal antibiotics include, e.g., griseofulvin, ketoconazole, itraconizole, amphotericin B, nystatin, and candicidin; antiviral agents include, e.g., zidovudine (AZT), amantadine hydrochloride, ribavirin, and acyclovir; antimicrobials include, e.g., cephalosporins (e.g., cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, cefadroxil, and cefuroxime sodium), penicillins (e.g., ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium), and erythromycins (e.g., erythromycin ethylsuccinate, erythromycin, erythromycin estolate, erythromycin lactobionate, erythromycin stearate, and erythromycin ethylsuccinate), and tetracyclines (e.g., tetracycline hydrochloride, doxycycline hyclate, minocycline hydrochloride, azithromycin, and clarithromycin); anti-infectives include, e.g., GM-CSF; sympathomimetics include, e.g., epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, epinephrine, and epinephrine bitartrate; anticholinergics include, e.g., ipratropium bromide, benzhexyl, trihexphenidyl, benzotropine, diphenhydramine hydrochloride, orphenadrine, chlorphenoxamine, amitriptyline, doxepin, imipramine, nortriptyline, biperiden, ethopropazine, procyclidine, cycrimine, and ethopropzaine; xanthines include, e.g., aminophylline, dyphylline, metaproterenol sulfate, and aminophylline; mast cell stabilizers include, e.g., cromolyn sodium; bronchodilators include, e.g., salbutamol, budesonide, ketotifen, salmeterol, xinafoate, terbutaline sulfate, theophylline, nedocromil sodium, metaproterenol sulfate, flunisolide, and fluticasone proprionate; androgens include, e.g., danazol, testosterone cypionate, fluoxymesterone, ethyltestosterone, testosterone enathate, methyltestosterone; estrogens include, e.g., estradiol, estropipate, and conjugated estrogens; progestins include, e.g., methoxyprogesterone acetate, and norethindrone acetate; adrenal corticosteroids include, e.g., cortisol, cortisone, oxandrolone, creatine, erythropeotin, dehydroepiandrosterone triamcinolone, betamethasone, betamethasone sodium phosphate, dexamethasone, dexamethasone sodium phosphate, dexamethasone acetate, prednisone, prednisolone, methylprednisolone acetate suspension, triamcinolone acetonide, hydrocortisone sodium succinate, triamcinolone hexacetonide, hydrocortisone, hydrocortisone cypionate, fludrocortisone acetate, paramethasone acetate, prednisolone tebutate, and prednisolone acetate; thyroid hormones include, e.g., levothyroxine sodium; antihypoglycemic agents include, e.g., human insulin, purified beef insulin, purified pork insulin, glyburide, chlorpropamide, glipizide, tolbutamide, and tolazamide; anti-lipidemics include e.g., antiatherosclerotics and antihypercholesteremics (e.g., cholesteryl ester transfer protein (CETP) inhibitors, such as those disclosed in U.S. Pat. No. 6,458,850; ileal bile acid transport (IBAT) inhibitors, such as those disclosed in U.S. Pat. No. 6,458,851; and HMG CoA reductase inhibitors, such as those disclosed in U.S. Pat. No. 6,462,091), fibric acid derivatives (e.g., clofibrate, fenofibrate, ciprofibrate, benzafibrate, clinofibrate, binifibrate and gemfibrozil), and nicotinic acid derivatives (e.g., nicotinic acid, niceritrol, and acipimox), dextrothyroxine sodium, probucol, pravastatin, atorvastatin, lovastatin, and niacin; antiulcer/antireflux agents include, e.g., famotidine, cimetidine, and ranitidine hydrochloride; antiemetics/antinauseants include, e.g., meclizine hydrochloride, nabilone, prochlorperazine, dimenhydrinate, promethazine hydrochloride, thiethylperazine, and scopolamine; collagen synthesis inhibitors include, e.g., prolyl hydroxylase inhibitors, C-proteinase inhibitors, and halofuginone; vitamins include oil-soluble vitamins (e.g., vitamins A, D, E, and K); amino acids include, e.g., valine, leucine, and isoleucine; proteins include, e.g., cyclophilin, antithymocyte globulin, immunoglobulin, muromonab-CD3, daclizumab, basiliximab, infliximab, etanercept, DNase, alginase, L-asparaginase, superoxide dismutase (SOD), lipase, metallothionine, a polipoprotein E, oxandrolone, creatine, dehydro epiandrosterone, platelet derived growth factor, fibrin, fibrinogen, collagen, interleukins 1 through 18, luteinizing hormone releasing hormone (LHRH), gonadotropin releasing hormone (GnRH), and transforming growth factor-.beta. (TGF-.beta.), tumor necrosis factor-.alpha. and .beta. (TNF-.alpha. and .beta.), nerve growth factor (NGF), growth hormone releasing factor (GHRF), epidermal growth factor (EGF), fibroblast growth factor homologous factor (FGFHF); hepatocyte growth factor (HGF); insulin growth factor (IGF), invasion inhibiting factor-2 (IIF-2), bone morphogenetic proteins 1-7 (BMP 1-7), somatostatin; thymosin-.alpha.-1, and .gamma.-globulin. Various biologically active forms of these proteins, including recombinant forms, mutants, complements, analogs, derivatives, and fragments are also contemplated. Other useful agents include nucleic acids (e.g., sense or anti-sense nucleic acids encoding any therapeutically useful protein, including any of the proteins described herein).

General Principles and Construction of the Invention

Normal operating conditions for the unit described herein for normal atmospheric conditions (temperatures and barometric pressure) as experienced in the home, office or laboratory. The normal operating conditions expected for use in this equipment would not apply to special conditions such as pressurized cabins or hyperbaric chambers.

The preferred construction is food or medical grade tubing and fittings assembled with no angled connections that can drive turbulence and disrupt the desired structured water at the output of the system. All tubing and fittings should be resistant to any corrosion or oxidation caused by chemical introductions into the system.

Depending on the application, quick disconnect fittings are desirable in any location where a change of the medium or component must be facilitated. Tubing and piping should be sized according to flow requirements required to manage both input and output flow, as well as any pressure accumulation found during changes in medium density.

Filters, shutoffs, valves, gauges, tanks and medium chambers are flow through exhibiting low resistance at lower water pressures.

Fittings, connectors, quick disconnects, and any other devices used in transitions of the water flow should consider flow-through approaches and as much is possible provide no extreme angles which would promote turbulence.

Similarly, frequency application and removal is done by passing tubing through or by the field emitter device without obstructing the water flow.

All piped components should be able to withstand operating temperatures typical for household plumbing applications, from 50° F. to 120° F. All components should be free of residues and or materials that may leach into the structured water makeup. When foodgrade plastics are not used, food grade stainless is recommended.

All electrical devices are recommended to be low-voltage, shielded when possible, rated for use in wet environments and appropriately NEC rated.

Sensors should be of a size, shape and type that will not promote inflow turbulence as well. Sensors should also be installed so as to avoid any EMF into the structured water. Sensors with metal tips should be foodgrade to avoid corrosion and contamination accumulation. Sensors systems should include capability for the use of disconnect from the piping for cleaning and periodic testing.

All electrical devices should provide outputs or indicators to report conditions or status.

Automation of components such as mixing devices may be considered provided the logic systems, whether commercial or proprietary, provide failsafes for detecting variations in pH or failures in operating devices such as the UV light.

Readout devices such as displays should display the accurate adjusted information. Critical changes in temperature may influence the desired state of the structured water at output and in the absence of an operator knowledgeable in such variations, displays should be programmable to adjust for such conditions changing during operation.

Electro/Mechanical Description

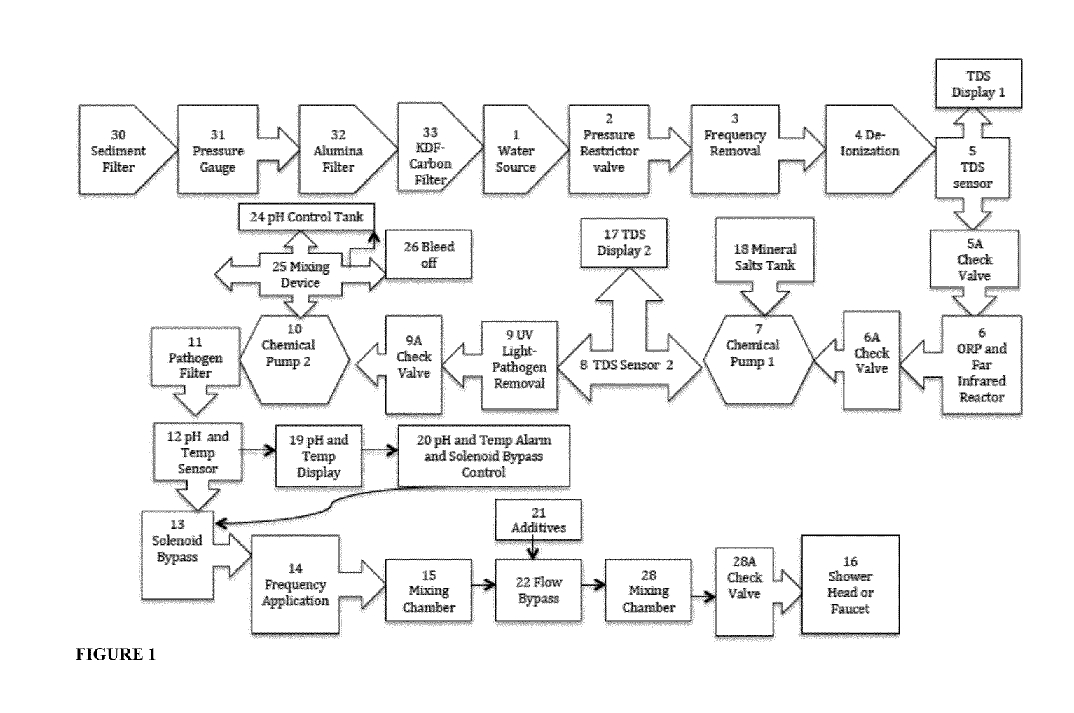

Referring to FIG. 1, the process mechanicals consider a water source where preliminary purification has already occurred. Typical pre filtering includes a sediment filter 30 such as a 5 micron poly wound fiber filter, an alumina filter 32 to remove arsenic, fluorine, selenium and other unwanted elements, a filter with KDF and activated carbon to remove clorine, heavy metals volatile components and to improve redox. The invention assumes the water to be free of solids, totally dissolved solids, volatiles, heavy metals, halogens, and other contaminants, organic and inorganic. Such filtration could be provided by a variety of conventional home systems or professional systems where water treatment has occurred through reverse osmosis or distillation.

The pre-purified water may be supplied via water heating equipment to assure the proper chemistry occurs relative to temperature reaction. Water sources may be in the form of holding tanks, direct piping connections, household plumbing fixtures, or other means to provide the water source. Appropriate shutoffs, nipples, connectors, or other devices to attach the water source to the invention may be supplied as appropriate to each application.

An appropriate pressure regulating device 2 provides for reduction of pressure, if required, from the water source. The invention typically utilizes industry-standard flexible plumbing tubing and quick connect disconnects such as John Guest, but is not limited to such tubing and fittings relative to plumbing hardware systems, and will operate best at pressures between 10 and 20 PSI. The regulation of this pressure in this invention is also to allow even flow through the various medium tanks and to reduce the potential for cluster distortion during turbulence. Typical plumbing is John Guest tubing and fittings or equal.

An optional device 3 may be supplied to treat the frequency of the sourced water. This process is not required, but may enhance the stability of the structured water produced. Invention may utilize magnetic, electromagnetic, electrical, audio, or other frequency emission devices to achieve specific outcomes for the water quality. An example would be an extremely low frequency (“ELF”) generator by Zephyr Industries or an equivalent. (http://www.zephyrtechnology.com/The_ELF_Generator/the_elf_generator.html)

A deionization filter 4 constructed of non-reactive plastic, commonly found in water filtration equipment, is used. The container is flow through with an input and output on the ends of the container so as to prevent turbulence while passing through. The medium used in filter 4 is a nuclear grade de-ionization resin. It should be a mixed bed type, appropriate for food and pharmaceutical applications.

An input TDS sensor 5 is used to determine if or not the de-ionization process of the water source is suitable for further treatment. A unit capable of visual display of total TDS or the unit capable of providing a mega ohm output to a central control device is preferred. The associated TDS sensor should be mounted within in-line bracket that allows to sensor tip to achieve a proper read at minimum turbulence in the tubing flow path. Rating output may be in parts per million as well as mOHMs. mOHMs should be 16-18 at input from the source. Typical is a DM-1: In-Line Dual TDS Monitor.

Check valve 5A is used either in-line or as part of the input nipple to the process six medium, and as a mechanism for assuring that no backflow occurs when source pressure is relieved or lowered. Typical is John Guest or equal.

A flow through media vessel 6 holds two types of medium. Past the input of the vessel, ORP resin fills approximately fifty percent (50%) of the vessel volume. FAR-infrared ceramic balls are mixed with the ORP resin accounting for the other fifty percent (50%). This combination establishes highly energized structured water and depending upon the resin used, should deliver −400 ORP or greater. Typical ORP medium is AIHENG model AH-FLZ. FAR Infrared is AIHENG model AH-FIR.

A check valve 6A is used either in-line or as part of the input nipple to the process seven mixing device as a mechanism for assuring that no backflow occurs when source pressure is relieved or lowered. Typical is John Guest or equal.

Optional mineral salts may be introduced to the structured water from a tank 18 via either an industrial chemical pump 7 or a section style needle valve. Metering of mineral salts from a liquid solution holding tank should be incremental and adjustable to allow for stabilizing the total TDS count at this point in the structured water. The types and TDS characteristics are application dependent and the use of certain mineral salts may cause chemical imbalance which affects the structured water. Flow pressure for the particular application dictates which type of mixing device is most suitable and is a question appropriate for each design and application. Electric metering devices which may create electromagnetic fields are not recommended as they may impact water structure if used in line. Typical is a Chemolizer HN series injector series pumps.

A second TDS sensor 8 is used to measure total dissolved solids. The mechanicals remain as in TDS sensor 5, however megaohms may read in the 100 to 120 range. Typical is a DM-1: In-Line Dual TDS Monitor.

An in-line flow through sterilizing light 9 is used and to ensure that any newly introduced materials in optional step seven or before have not placed pathogens into the water mix. It totally contained UV light is recommended with broad-spectrum kill capability and indicator lights to allow the operator to observe the light is operating. Materials used should be stainless or other nonreactive materials typically found in foodgrade version UV lights. Typical is Sterilight rated 40 mJ/cm2.

A check valve 9A is used either in-line or as part of the output nipple from the sterilizer as a mechanism for assuring that no backflow occurs when source pressure is relieved or lowered.

A holding tank of pharmaceutical or food grade ascorbic acid which has been premixed with structured water at a pH of approximately 3.0 is brought in through the mixing device 25 into the flow. Incremental metering changes must be made to allow regulating and monitoring the pH of the structured water.

The water flows through a vessel 11 containing pathogen filtration materials having a filtration capability of 0.5 micron or less to further assure no pathogen is able to be transferred to the output. Typical is Berkfield Super Sterasyl™

pH is monitored by an in-line pH and temperature sensor 12 which provides information on the total pH produced after final mixing. In applications where no manual operator is in attendance of the equipment, the pH and temperature sensor should have a closed system logic with an output capable of driving a solenoid operating a bypass valve that can divert any out of range pH structured water to a drain. Typical is an ATI model Q45.

A solenoid bypass valve 13 operates in a normally open position. The valve 13 in its normal open position, the flow should remain unrestricted and operating in a 180° flow path. In three way valves or four-way valves any angle there are 90° flow changes should be limited to the bypass mode. The valve is a safety valve set to divert when activated via pH sensor 12. Typical is ASCO or equal.

A second frequency modulator device 14 may be used to apply a frequency or frequencies to the structured water. This device would have the same characteristics as frequency modulator device 3.

An in-line mixing chamber 15 is added to the main line along with a related bypass. The chamber operates as a removable vesicle in which structured water and water soluble topical supplements may be added. Typical is a Watts filter housing or equal. An example might be a food grade vitamin C powder. Suitable shutoffs which smoothly very the flow to the mixing chamber or alternately to the bypass allow the control of the amount of supplement which is applied to the output. The same shutoffs also provide pressure restriction at such time as a new supplement or nutrient is desired.

A final check valve 28A is placed in line were appropriate to allow the flow to pasture normally under normal working conditions. The valve should be located as an attachment to the showerhead or output nozzle to prevent ambient air from entering the line.

An output device 16 such as a faucet, nozzle or showerhead should be installed to provide metering for the structured water onto the person or object. The unit may or may not require a shutoff feature.

Using the water of the present invention, it is possible to treat nutritional deficiencies or deliver therapeutic agents through the skin using the water alone or with an enhancing agent. By providing a convenient and effective transdermal absorption process using water uptake during the showering or bathing process we can encourage better natural hydration of the body and its cells. To achieve the desired degree of uptake requires that the water have certain properties to induce transdermal absorption as well as be free of non-beneficial properties. Typically, structured water processes that are available today produce the water via electrolysis. Electrolysis generally produces ionized water that is alkaline in nature and which at high ORP may represent an alkaline value that would be resisted for transdermal uptake by the body and would dangerous to skin or underlying tissues.

Further electrolytic cells are cost prohibitive and cannot control ORP and pH separately to get a safe, vicinal grade water at a biologically acceptable pH with the desired high-ORP that can be absorbed transdermally without causing secondary pH neutralizing reactions. The invention described herein eliminates the need to electrically convert water molecules which at high alkaline or acid pH production creates inverse by-products that must be handled as wastewater.

The goal of the present invention is to provide a bioavailable, structured ionic water that is pH perfect and high negative ORP at a reasonable cost for a typical household bathing or showering application. In addition, the water would be free of any negative characteristics. It is an object of the invention to generate water which is 400 negative ORP or greater, normally at a vicinal pH of 7.35 to 7.45, free of any organic or inorganic impurities, free of all pathogens, and having a very small water cluster size that the body could use immediately in interstitial fluids and subsequently in aquaporin uptake into the cells.

To achieve the desired results a process was developed which purified and chemically changed the water. This process achieved water which has the following properties

a. High negative ORP (approximately −400 mV) while at 7.35-7.45 pH.
b. H2O cluster of <6.
c. Not using electrolytic process to ionize or structure the water.

This process relies on the use of infrared radiation, more specifically the infrared radiation supplied by tourmaline.

An antioxidant's potential to supply electrons dispersed into a liquid can be measured by using an ORP (Oxidation/Reduction Potential) meter. Oxidized materials are shown as + above zero, antioxidants are either a low + or a negative reading. Lower numbers indicate more available electrons. For example, the antioxidant CoQ10 has an ORP of +49 mV; wheat grass juice has an ORP of −120 mV. The negative reading of wheat grass juice gives it a significantly higher potential for donating electrons and neutralizing free radicals than CoQ10.

As described further herein, the water of the present invention has the ability to neutiralize —OH and other electron seeking free radicals. It has high bioavailability for uptake through the skin and cellular aquaporin intake based on optimal pH and cluster size. It does not contain highly acidic or alkaline byproducts which usually result from ionization processes.

It is an object of this invention to produce a structured water cluster size less than six molecules as being highly bioavailable. Because of the tendency of water molecules, as normally found in tap water to be a cluster of 12-16 or greater, the effectiveness of water in hydrating the body when applied to the skin is limited. This invention consistently provides a very small cluster size of less than six and during the initial NMR test sequence measurements, the water quality was shown to be 40.4 Hz. which would indicate a cluster size of more on the order of 4, which is optimal for flexibility in bonding and bioavailability. Comparative numbers in hertz is would show tap water that about 120 Hz for around 12 water molecules per cluster, Evian spring water at 82.6 Hz and popular structured water brands found in supermarkets at approximately 63 Hz. which is considered hexagonal water or a cluster size of 6. This cluster characteristic in this invention supports the waters use as an interstitial fluid equivalent for hydration of cells.

This embodiment provides optimal conditions for hydration by supplying structured water closest in characteristic to interstitial fluid. The mechanisms for cellular hydration, aquaporins, are varied and act as gatekeepers to intracellular space. When water absorption is improved by reducing the cluster size, hydration can speed up. In in doing so, transdermal absorption can be optimized. Kazuyoshi Murata, Kaoru Mitsuoka, Teruhisa Hirai, Thomas Walz, Peter Agre, J. Bernard Heyma, Andreas Engel & Yoshinori Fujiyoshi Structural determinants of water permeation through aquaporin-1; Nature: 407, pp 599 (Oct. 5, 2007).

It is an object of this invention to create a water that is equivalent to vicinal fluid, that is lacking in any impurities and being identified as only water molecules in <4 molecule clusters, found in intracellular spaces to maintain ideal pH for extra and intracellular hydration in the range of 7.3 to 7.5 while delivering in ORP measured as −380 mV or higher. Under normal conditions this relative measure would not be possible using technologies such as electrolysis. High negative ORP rarely is exhibited except when pH is over 9.0. This ability to maintain free hydrogen at this low, bio-available pH allows the structured water to remove free radicals in the interstitial fluid areas after passing into the skin.

It is an object of this invention to reduce acidosis. The ability to hydrate with the pH 7.3 to 7.5 supports a reduced need for the production of bi-carbonates in the bloodstream. This ideal pH allows for hydration to substantially reduce potential acidosis, particularly in older individuals who are less able to produce by carbonates.

It is an object of this invention to deliver bioenergy to cells. By exhibiting high ORP the invention supports bio-energetic revitalization of cellular voltages, as shown in bodily organs where measurements for the liver show −170 mV, large intestine −250 mV, small intestine −150 mV stomach, −150 mV and rectum −200 mV. The invention's ability to produce vicinal equivalent water with H ions in a stable cellular pH can provide bioavailable energy that the cell can use immediately or store. See Murata, supra and Nutritional Supplement by Hydrid Ions acting as Antioxidants and Hydrid Ions and H Atoms as Energy Currency for Living Systems by Prof Dr. Randolph Riemschneider, Institute of Biochemistry, Free University (FU) Berlin, Germany and Central Institute of Chemistry, Universidade Federal de Santa Maria (UFSM), Santa Maria, Rio Grande do Sul, Brazil. http://www.bwwsociety.org/journal/html/hydrid.htm

It is an object of this invention to provide water having a reduction capability as an antioxidant. It is well understood that a wide array of free radicals are present in the interstitial and intracellular fluids resulting from environmental exposure. In most tissue such free radicals are responsible for deterioration of chemical reactions and more importantly, the destabilization of amino acids, in particular RNA and DNA. Balancing redox reactions in vivo can significantly improve cellular health. See Scalar Structured Water test reports. http://sacredscalarenergy.com/scalar-structured-water/23.html Indications of NMR readings for hexagonal water as well as impedance tests and phase angle tests all providing supporting evidence of improved cellular health benefits.

It is an object of this invention to deliver nutrients. High negative ORP value provides greater opportunity for the structured water to maximize the delivery of nutrients in vivo. Nutrients of all kinds that are dependent upon proper stereochemistry to be acknowledged by cellular receptors can be most readily accepted when the medium carrying nutrients allows for nutritional molecules to be readily recognized and utilized at the cellular level. By improving hydration and creating neutral conditions for interstitial fluid, as well as providing an anabolic environment supports cellular uptake of nutritional molecules. See, Structured Water Test Reports, supra.

It is an object of this invention to improve cellular health. Treated cells have the ability to more rapidly remove cellular wastes from the intracellular gradient to the extracellular gradient. Studies in the area of hexagonal structured waters have reviewed cellular phase angles and bio-impedance and found that reduced cluster size does allow for more rapid and efficient cellular exchange and eventual lymphatic elimination. See, Structured Water Test Reports, supra and Role of Water in Some Biological Processes, PHILIPPA M. WIGGINS, Department of Medicine, University of Auckland School of Medicine, Private Bag, Auckland, New Zealand. VOL. 54, 1990.

It is an object of this invention to improve the quantity of protein production by hydration, The embodiment supports studies on the importance of hydration in anticodon loops of tRNA. The implications are that small cluster size and bioavailability of structured water produced in fact help manage successful protein production as result of tRNA hydration improvement. Further, transcription improvement is also cited for DNA as the result of hydration with structured water. See Biochim Biophys Acta. 1979 Nov. 22; 565(1):131-47. Theoretical study of hydration of RNA. Kim K, Jhon.

It is an object of this invention to deliver plant nutrient molecules that can influence cellular health by providing miRNA that can improve cellular health. In the paper, “Exogenous plant MIR168a specifically targets mammalian LDLRAP1: evidence of cross-kingdom regulation by microRNA” Cell Research (2012) 22:107-126. doi:10.1038/cr.2011.158; by Zhang, how, Chen et al clearly shows the rapid LDL changes in mice resulting from closely monitored study of MIR168a.

It is an object of this invention to apply structured water's ability to remember frequency signatures of organic molecules that have been in the water. Clear identification of techniques provide the ability to determine and apply phytochemical frequency signatures. Applying such water memory borne frequencies may induce intracellular processes in the same fashion as the physical presence of phytochemicals bearing those frequency signatures. Refer to ‘Phytochemical Techniques” N. Ramaan, 2006 ISBN 81-89422-30-8.

It is the object of this invention to apply structured water's ability to remember frequency signatures of organic molecules that have been in the water to create positive cellular activities by utilizing water based frequencies.

It is the object of this invention to apply structured water's ability to remember frequency signatures in developing a medium to manage pathogens in the medium by ablating pathogenic frequencies. Extensive work in recognizing pathogens was done by Dr. Hulda Clark in this field. Refer to her list of mold, bacteria and virus frequency signatures found in “The Cure For All Diseases: With Many Case Histories”, Copyright 1995 by Dr. Hulda Regehr Clark.

It is an object of this invention to deliver plant nutrient molecules that can reduce inflammation. Refer to “Inflammation and You: How Foods From Plants Protect Us From Disease” was published in the April 2009 issue of Agricultural Research magazine. The supporting work was from Zhao L, Lee J Y, Hwang D H “Inhibition of pattern recognition receptor-mediated inflammation by bioactive phytochemicals: A review of recent research”. Nutr Rev. 2011 June; 69(6):. doi:10.1111/j.1753-4887.2011.00394.x.

Without limiting the scope of the invention, the inventors believe that structured water of the present invention operates to translocate nutrients and pharmacologically active agents into the body, organs and cells by iontophoretically transporting molecules without the need for external electrical fields as well as deliver water memorized frequencies that impact cellular health.

It is an object of the invention to use structured water orally in mixing with various supplements, micronutrients and beneficial molecules such as naturally occurring enzymes to improve the quality of nutrient uptake and post celiac absorption. Such uses could include premixes in the form of beverages, used as a means to mix into solution dry nutrients such as freeze-dried powders, as well as the use of those solutions in various food preparations.

It is an object of the invention to use structured water early in mixing with concentrates to improve uptake other than post celiac. Such solutions could be applied to sublingual applications as well as nasal sprays or rectal infusions.

It is an object of the invention to create structured water solutions for oral use in the production of capsules, gels, pills, salves, lozenges, tablets, powders, infusions, solutions, beverages, and other products consumed by mouth.

It is an object of the invention to use the structured water when orally taking pills or capsules to improve the quality of digestive up take and absorption.

It is an object of the invention to use the structured water as a means of oral hydration and natural energy replenishment.

It is an object of this invention to use structured water in beverages to enhance flavors and improve nutrients in the beverages.

It is an object of this invention to improve compounding processes using structured water.

### Example 1

### Device and Process for Making Structured Water

A device was constructed embodying the principles described above to convert tap water to structured water. Both the selection of materials and the piping design are critical in achieving and maintaining cluster sizes. This embodiment allows pH and negative ORP to be separately determined.

To create the solution, an available source of potable water is delivered to the equipment and the flow pressure regulated. This water is ultrapurified into the megaohm range using sediment, alumina, carbon and ion exchange resins. The design of the piping and plumbing is constructed to prevent turbulence that could alter cluster characteristics. Radiant devices, in lieu of chemicals, are added to assure pathogens would be eliminated from the water. Referring to FIG. 1, the following processes are a specific sequence of chemical, hydraulic, sensory, electro/mechanical, and radiant technologies performed in sequence to create, protect and preserve the resulting structured water. While the processes below are performed on water in the liquid state, the construction of the invention may be suited to allow for the final application of the structured water to be in any normally existing state of water.

Process 1—Creation of Ultrapure Water from Source Water.

Referring to FIG. 1, water from a source 1 is fed into the system. The water can be any source of water which is suitable for consumption, including, but not limited to municipal tap water. Depending on the source water, it may be necessary to filter the water to remove particles. In the present embodiment a 5 micron in line sediment filter 30 is used. Pressure should be monitored to ensure adequate pressure after filtration. This is preferably achieved with a pressure gauge 31.

Following sediment removal, the water is passed through an alumina filter 32 to remove contaminants such as fluorine, arsenic and selenium. The water is then passed through a third filter 33 containing copper-zinc granules (KDF 55 from KDF Fluid Treatment, Inc) and activated carbon to remove chlorine, soluble and insoluble heavy metals, hydrogen sulfide, ferrous iron, and volatile compounds.

Depending on the flow and pressure capabilities of the system, a pressure regulator 2 can be installed. In the present embodiment, pressure was restricted to approximately 15 PSI.

Optionally, any undesired frequencies are removed from the water via a frequency modulator 3. Such frequency modulators are known in the art and include the ELF generator by Zephyr Industries or an equivalent.

The water is next de-ionized by passing it through a deionization filter 4. In the present embodiment, the deionization media is NRW-37 from Purolite. This assures all mineral ions are removed, such as cations from sodium, calcium, iron, and copper, and anions such as chloride and sulfate.

The above processes, if performed within the flow and pressure specifications of the component filter manufacture, will provide water which is at from 1 megohm to 18.3 megohms in purity. Water below 1 megaohm purity is inadequate. A quality check can be performed using an optional total dissolved solids (TDS) sensor 5 which displays on TDS display 27 The reading should be zero.

If the water source is sufficiently pure, (greater than one megaohm purity) one or more steps of process 1 may be omitted.

### Process 2—Production of Structured Water

Still referring to FIG. 1, water of at least one megaohm purity is passed through a check valve 5a and fed into a reactor 6 containing a mixture of ceramic media capable of lowering the ORP to at least −400. In the present embodiment, the ceramics are in marble form and release over −400 mV plus of H and provide a high rate of ionization as well as radiating 4-14 micron band photons of light energy in the into the water.

The media comprise tourmaline ceramic marbles which reduce the cluster size of the water molecules from 20 to 5 and far infrared ceramic marbles which emit far infrared radiation. A suitable Tourmaline ceramic marble is the thirty percent (30%) tourmaline marble sold by Pingxiang Nanxiang Chemical Packing Co. Ltd. under the brand name: NX-FIEB http://www.nxpacking.com/product/html/?54.html

To prevent contamination between the processing steps and to eliminate leakage on disassembly, check valves are used.

Optionally at this stage the water may be remineralized. By injecting dissolved minerals from mineral salts tank 18 via a pump 7. Any purified salts may be used. In one embodiment purified salts such Himalayan salts are added to provide mineral based health benefits to the structured water.

Total dissolved solids are checked again using a sensor 8 which displays on display 17. Pathogens are removed via UV light 9. Optionally pathogens can be filtered using ultra fine filtration media suitable for pathogen removal. An option check valve 9A is provided to prevent backflow.

The water resulting from the previous processes will tend to be too basic due to the high ORP. A solution of acid, such as ascorbic acid is added via a pump 10 from control tank 24 to bring down any pH readings above about 7.45. Water not meeting the pH spec is bled off via return line 26

In a final purification step, the water is passed through a pathogen filter 11 having a size no greater than 0.5 microns to assure that any microbes are physically removed.

A pH sensor 12 monitors the water in real time and will activate a drain valve if the water is not within the accepted pH range of 7.0 to 8.0. Optionally, temperature is included with the pH sensor. The sensor data is shown on display 19. If the pH sensor sees an out of range read and the water must be sent to a secondary drain, the by-pass valve 13 is energized from the control board logic 20

The water resulting from this process is pure and suitable for use for bathing and drinking.

Process 3 Further Enhancements

The water from Process 2 can be further enhanced to provide nutritional or pharmacological benefits. In one embodiment, the frequency of the water is adjusted with a frequency generator 14.

In another embodiment, the water is mixed with additives such as dietary supplements, homeopathic agents or pharmacologically active agents via mixing chamber 15. The additives are placed in tank 21. A flow bypass control 22 controls whether or not additives are mixed with the water. Water containing additives is stored in mixing chamber 28. Mixing chamber 28 is connected to a delivery means 16 such as a shower or faucet. Optional check valve 28A can be used between chamber 28 and the delivery means to prevent back flow.

### Example 2

### Delivery of Structured Water

The structured water developed in Example 1 can be administered via any conventional means. While it has activity if consumed orally, it is most preferred to administer the structured water topically to avoid any degradation by the digestive system or first pass effects of the liver.

In one embodiment, the structured water is delivered to a shower head or faucet for bathing.

In another embodiment the structured water is applied using a sponge, cloth or compress.

In yet another embodiment the structured water is used as an ingredient in mixing/making products for oral consumption or for topical application.

In another embodiment, the water is used for intravenous, intramuscular or intraperitoneal use.

In another embodiment the water is used for dialysis.

In yet another embodiment the water is delivered via a pulmonary route with or without additional agents added.

In yet another embodiment the water is used for wound or burn care.

In yet another embodiment the water is used for delivering naturally occurring Humic and Fulvic acids as a means to provide their inherent anti-pathogenic properties such as immune support and chelation.

### Example 3

Natural penetration enhancers exist which are complementary to the present invention. Such enhancers include humic acids and fulvic acids which act as biologically friendly surfactants. See, Randhawa G K, Kullar J S, R. Bioenhancers from mother nature and their applicability in modern medicine. Int J App Basic Med Res [serial online] 2011 [cited 2013 Mar. 14]; 1:5-10. Available from: http://www.ijabmr.org/text.asp?2011/1/1/5/81972 Biologically acceptable surfactants may also be used to improve penetration. Surfactants are compounds that lower the surface tension of a liquid, the interfacial tension between two liquids, or that between a liquid and a solid. Lecithin is a type of fat produced naturally by plant and animal life, humans included. The substance can be found in various consumer products, not for flavor but as an emulsifier, or gluing agent, to keep the ingredients from falling apart. Also known as phosphatidylcholine, lecithin is typically derived from soybeans and can be found in products as divergent as mayonnaise, chocolate, moisturizer and baby formula.

Humic and Fulvic acid enhancers present valuable anti-microbial properties in structured water. Refer to An in vitro investigation of the antimicrobial activity of oxifulvic acid”, CEJ Van Resberg, A. Van Shaten, J. Dekker, “2000—46-853-4, Journal of Antimicrobial Chemotherapy. And “Investigation of the Anti-HIV properties of Oxihumate” C E J Van Resberg, A J. Dekker, R. Weis, T.-L Smith, Janse vanRensberg, J. Schneider, Chemotherapy 2002, 48:138-143 (DOI:10.1159/0000064919.

### Example 4

Negative ORP water has the ability to induce higher metabolic activity through introduction of hydrogen ion energy and free radical reduction as a result of hydrogen ions presence in interstitial and intracellular fluids. Testing was performed for specific biomarkers and changes pre- and post-test. Blood pressure, blood glucose and pH improved in the test subject. Three summary points taken from that test are as follow:
Blood Pressure—Supine systolic (laying flat resting) blood pressure was reduced by 4.5%
Blood glucose level dropped 15% showing increased high and immediate cellular activity which increased demand for available glucose.
Salivary pH—pH improved from 5.5 to 6.0 showing a reduction in acidity or acidosis.

Further observations of the researcher noted that it would normally take 2-3 weeks of therapy to achieve the fatty acid composition changes seen. It was hypothesized that the drop in blood glucose levels were due to increased metabolic activity.

Visual Contrast Sensitivity (VCS) testing on the subject showed marked improvement.

The visual system includes a complex neurological network that involves the retina, optic nerve, brain nuclei and the visual cortex. One of the main outputs of the visual system is pattern vision. The VCS tests is known in the art and is an indicator of ability to detect visual patterns. See, U.S. Pat. Nos. 4,365,873; 5,414,479 and 5,500,699. The test measures the least amount of contrast between light and dark bars (sinusoidal grating) that is needed for the viewer to detect the bars. VCS is measured at five different bar sizes (spatial frequencies) because perception of different bar sizes is mediated by different physiological components, and these components are differentially susceptible to effects from different toxic substances (10-17). The largest effects of biotoxins are at the mid-size bars (1-8). To measure VCS, viewers are presented a series of bar patterns at each of the five bar sizes. Viewers respond by indicating that the bars are tilted to the left, tilted to the right, are straight up and down, or that they cannot see any bars. The pattern with the lowest contrast that is correctly identified is the measure of VCS for that bar size. Upon completing the VCS test, viewers receive a message indicating that biotoxins are (positive) or are not (negative) likely to be involved in their illness. The criteria for getting a “positive” VCS result is set high to avoid false positive results. This occasionally results in a false negative result; some cases of chronic-biotoxin induced illness may pass the VCS test some times. VCS can be measured during treatment to monitor recovery.

### Example 5

### Bioelectrical Impedance Test

In vivo testing following the methods of Stephanson et al., supra, showed a 0.5 L move of intracellular fluids to extracellular. This would indicate that hydration occurred and that significant material, wastes in water, moved to the interstitial fluid surrounding the cells. Normally, bio-osmosis alone would not exhibit such a rapid change. Also, in conjunction with the toxicity, these changes showed significant hydration with the bio-energized water (hydrogen ions) had entered the cells via aquaporins and that the bio-energy levels intra-cellularly had received enough −mV to power the cell's proton pumps which will operate against the normal osmotic properties if the cells must evacuate waste materials.

### Example 6

### Measurement of Water Cluster Size

The art contains many ways to measure water cluster size including spectrographic and magnetic resonance methods. In preparation for using the invention, NMR tests were performed on the water to determine if any impurities, organic or inorganic showed up in the spectral group from the test water and to ascertain the cluster size based on the signature. Referring to Table 1 below, resultant data from the test using a Varian Oxford AS 400 Mercury plus NMR showed no impurities and a 40.4 Hz which is considered to be below hexagonal water. The hydrogen bonds are volatile and changing rapidly. Wikipedia's reference to water bond stability states “The molecules of water are constantly moving in relation to each other, and the hydrogen bonds are continually breaking and reforming at timescales faster than 200 femtoseconds”. The 40.4 Hz signature is showing a relative banding stability of a probable cluster size of 3-6 though 4-6 is more in keeping with conservative approach.

TABLE 1
NMR results.

| Test - NMR @ | | | | |
|---|---|---|---|---|
| pH 7.3 Sample B | value | measure | timeframe | notes |
| particulate matter | 0 | stated | immediate | no reported |
| | | microscopic | | non O2 spikes |
| | | grid | | |
| total dissolved solids | 0 | mOhms | immediate | no reported |
| | | | | non O2 spikes |
| ORP—oxidation | −380 | mVolts | immediate | by test meter |
| reduction potential | | | | from sample |
| water cluster size | 4 to 6 | molecules | immediate | 40.4 Hz |
| | | per cluster | | |
| spectrography - IDs of | 0 | chart report | immediate | no reported |
| non-water molecules | | | | non O2 spikes |

### Example 7

### Subject 1

A 51-year-old male was tested using the structured water. The test protocol concentration was 5% delivery, or 1 mL of a 1 oz preparation which contains 10.5 g (10,500 mg) of Pure Whole Live Marine Phytoplankton which is a delivery comparable to what might be an oral supplement dosage, and conservative to avoid any complications with test subjects. No inorganic material or pharmaceuticals were applied.

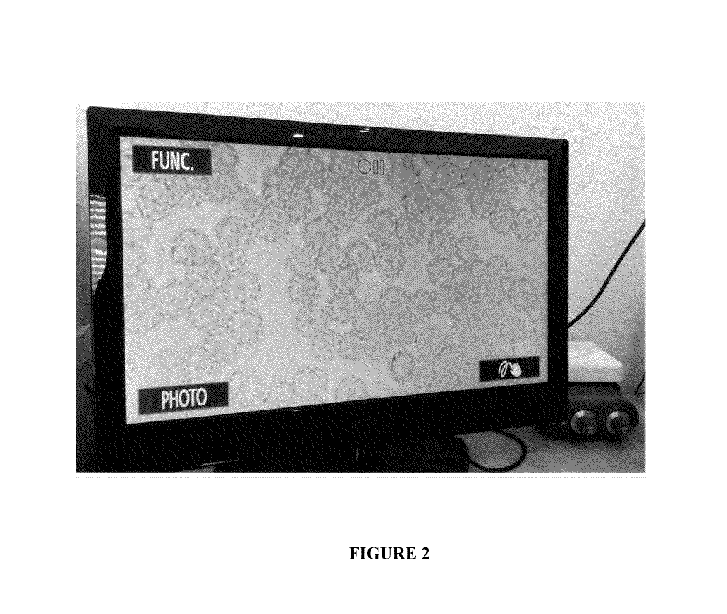

FIG. 2 is a photograph of the subject's blood before testing. In the before photograph well there is no significant signs of Rouleau, the overall condition of the red blood cells shows a unbelievably high stress exhibited in the form of each and every individual cells exhibiting dozens of stress markers. While it was unclear as to the source of the stress, the chemical conditions apparently present in the blood at the time would've let us to believe that the capability of the blood to deliver oxygen and even an environment for the plasma to be maintaining levels of nutrients would have been remarkably low. Moreover, the presence of typical immune cells, such as white blood cells and neutrophils were essentially absent. Very little motility was seen.

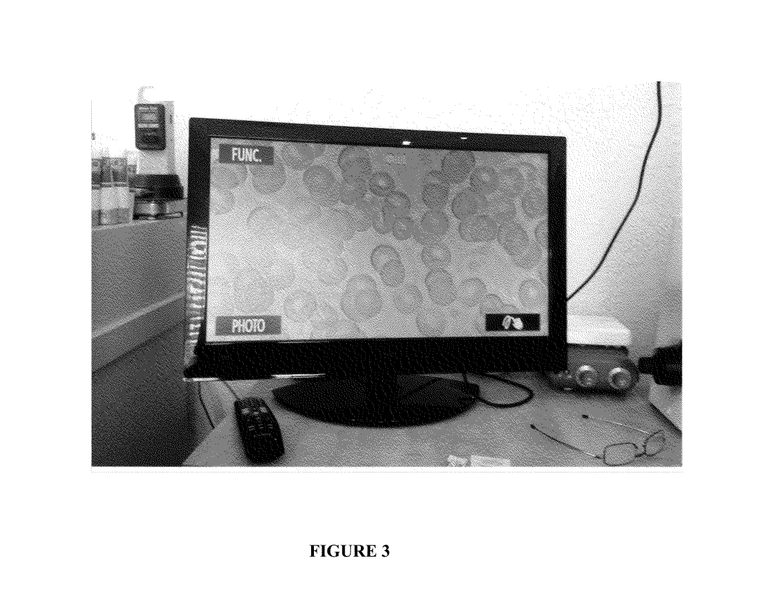

FIG. 3 is photograph of the subjects blood taken after the first test session. The after photographs exhibited a remarkable change. Motility and a fairly substantial level of bioenergy were apparent. Red blood cells appeared to be now shaped normally with virtually no stress markers present. Immune system cells absent prior were not present and appearing as motile as the red blood cells. In no other subject tests has such a dramatic change been as apparent.

The same 51-year-old male remained in the test program. An ongoing program was introduced to the transdermal approach to determine its effectiveness in the fatigue condition experienced by the test subject. Structured water was used in combination with pharmaceutical grade fulvic acid and a variety of organic freeze-dried high ORAC fruits and vegetables. The intent of developing a nutrient rich supplement was to provide the highest possible bioavailability of concentrated micro-nutrients in solution. The test subject indicated that within 30 min. to one hour he was able to experience significant energy with the symptomatic fatigue rapidly leaving and remaining at a low level until nearly a 24-hour period test.

Simple dark field microscopic evaluations of before and after the use of the structured water with nutrient mix showed that the total condition visible in blood serum improved markedly relative to reduction in Rouleau and improved motility in both red and white blood cells. Use over several days exhibited reductions in visible pathogens in the serum. The implication was that the oral use of high ORAC freeze-dried material and pharmaceutical grade fulvic acid provided in structured water improved bioavailability. It is further believed that post celiac rate of absorption and retention phytochemical molecular structures is higher based on previous test observations where similar materials tested transdermally with structured water showed rapid changes in blood samples before and after exposure.

Testing over a two-year period using various combinations of high ORAC fruits and vegetables as well as mushrooms appears to have significant impact on cellular epi-genetics which corresponds to many currently produced studies which examine the impact of plant-based phytochemicals and RNA in dramatically changing cellular phenotype.

### Example 8

### Subject 2

A 44 year old female test subject was tested for the ability to improve nutrient levels using the structured water of the present invention as a means to transdermally deliver a test group of vitamins. Medical profiling showed the subject to be very physically healthy, exhibiting exceptionally good biomarkers in generally accepted laboratory tests performed to determine deficiencies. The test protocol, would indicate cellular uptake of Vitamin B12, folate or folic acid (vitamin B9) and vitamin D3. A dermal sensitivity test was performed to assure no allergic reactions were possible. Homocysteine, a biomarker that is a recognized secondary reaction biomarker to correlate the effectiveness of the B9 and B12 absorption was evaluated as well. As all four of the selected biomarkers were measurable in standard laboratory blood plasma testing, the protocol provided a simple test that could be easily measured using a baseline, i.e. before test, and subsequent post-test measurements at reasonable intervals.

1000 mg pharmaceutical grade B12 and 4000 IU of vitamin D3 together with 20,000 mg of pharmaceutical grade humic acids were compounded in 0.25 L of structured water. No folate or folic acid was included in the compounding process as no suitable folic acid or folate were available.

The vitamin mixture was added in its entirety to the mixing vessel used in the structured water treatment system. The water treatment system was then used to deliver the vitamins diluted in the structured water using a metering mechanism. The subject showered in the structured water/vitamin solution at a temperature of approximately 38 degrees Celsius at a flow of approximately 2-3 gallons per minute.

### Biomarker #1—Folate (Vitamin B9).

Observed Measures—

a. baseline—12.6 ng/mL
b. at 45 minutes—13.2
c. at 1:15 hours—15.6

This result was considered highly unusual and unexpected as no folate was delivered by the structured water. A prior test performed in this subject showed a higher folate level than was observed on test day. The implication of the observed rise in folate is that the delivered bio-energy in the structured water test triggered a folate release at the organ (liver) or potentially, via intestinal flora.

Determination of relative comparative measures—in order to separate conjecture from factual elements, we required a resource reference document that allows us to evaluate whether or not the observed changes from the baseline to 45 min. after test application and again 75 min. after test application are significant. Researching a variety of white papers on folate absorption, several conclusions were probable, based on the paper found to best match the needed performance measures used in the test labs, as follow:

A 2009 article in the American Society for nutrition magazine referenced as Susanne Aufreiter, et al, Folate is absorbed across the colon of adults: evidence from cecal infusion of 13C-labeled [6S]-5-formyltetrahydrofolic acid, AM J Clin Nutr (2009),—90:116—23 served to provide the needed information. This paper provides a relative pharmacokinetic table reference as it compares absorption rates for 5-formyltetrahydrofolic acid, both intravenous and cecal infusion, expressing them in nmol/hr. This reference can be directly compared to our laboratory results of the baseline, and the 45 min. and 75 min. serum test results. The reference origin of the folate was via IV and alternately via cecal infusion; effectively as shown in the referenced comparative study, these are apples and oranges comparisons as the bio-availability of the cecal dose (average of 8.57% in the study or 2.14% when adjusted to volume versus the IV dosage), as used, is far lower than the IV dose, even at a 4× volume of sourced material compared to the IV dose. None-the-less, we did provide for an extrapolation for the comparisons in this report.

Test result conversion—to relate the folate test results and compare them to the pharmacokinetic table required that the nanograms/per milliliter should be directly compared with the nanomoles used in the reference table. Using a clinical conversion factor for folate ng/mL was multiplied by 2.266. On that basis, the test subject's serum readings could be converted.

Baseline—12.6 ng/mL=28.688 nmol
Post A @45 min.—13.2 ng/mL=29.911 nmol
Post B @75 min.—15.6 ng/mL=35.35 nmol

The rise in folate levels are comparable to intravenously applying 172 nmol of formyltetrahydrofolic acid—when the nmol conversion is evaluated against table 2 of Aufreiter (2009), pharmacokinetic data, and the rate of appearance column for intravenous injection is evaluated using the subject group mean+/−SEM (mean) value of 7 nmol/hr.

The rise in folate levels are also comparable to cecal infusion of 684 nmol of formyltetrahydrofolic acid. As in the intravenous data, the group mean was used. In this case, 0.6 nmol/hr rate of appearance would indicate a far lower rate of uptake occurs gastro-intestinally.

Based on the pharmacokinetic table's accuracy in the referenced 2009 clinical test and the accuracy of the conversion from nanograms per milliliter to nanomoles, the conclusion would indicate that structured water drove an unexplained increase in Folate. That increase was equal to intravenous and many times higher than the cecal infusion stated above in the reference test white paper without any introduction of folate or precursors. This leads to a hypothetical explanation that the structured water caused a liver release that may have been due to the body's recognized deficiency in Folate that was driven by fasting and upon an increase in cellular metabolic activity driven by the available bio-energy supplied to the cells during the test. A significantly less likely, but possible explanation, would exist which is the potential of the intestinal flora being bio-energized and synthesizing the serum folate increase. This seems unlikely in light of the previous levels being high and that liver release is a far more probable outcome.

### Biomarker #2—Vitamin B12

Observed Measures—

Baseline—663 pg/mL

at 45 min.—736

at 1:15 min.—648

It would seem the demand for B 12 had increased significantly after the test baseline and was actively being metabolized at the 45 min and 75 min. intervals. A pre-test results showed 1461. With the baseline result after the subject's cross country test, combined with the 24-hour plus fasting, indicates that stress was a likely cause of the significant reduction in B12. To create a means to evaluate the results, the following research was done.

In order to separate conjecture from factual elements, we required a resource reference document that allows us to evaluate whether or not the observed changes from the baseline to 45 min. after test application and again 75 min. after test application are significant. Researching a variety of white papers on B12 absorption, several conclusions were probable based on the paper found to best match the needed performance measures used in the test labs, as follow:

The publication entitled “Plasma Absorption of Cyanocobalamin Co57”, credited to Alfred Doscherholmen, M.D., PhD and Donna Ripley, taken from the archives of internal medicine, volume 134, December 1974 pages 1019 to 1024, was used as a reference document to establish performance rates that could be compared the hospital laboratory reports being used in the testing. Doscherholmen found on page 1022 that B12 provided a plasma absorption in pg/mL the directly related to the tests performed by the present test laboratory.

Doscherholmen in Table 2, showed that normal subjects exhibited an average of 8.0 pg/mL per our absorption. The range seen was from 3.2 to 15.1. If the average is used, the results of the change from the baseline, pretest, to the 45 min. result would indicate a 73 pg/mL increase, which is a nine times increase over the average were five times increase over the peak range seen in the 42 person subject test. This test result would confirm that the structured water was able to significantly increase serum levels of B12 over a known test group performed under controlled conditions.

Biomarker #3—Vitamin D3 (Tested D25 Hydroxy)

Test Measures—

Baseline—58 ng/mL

at 45 min.—60

at 1:15—59

The subject's pre-test dated showed that the test subject's D3 was well within the reference standard of 30-100 ng/ml. at 59.2. That condition being well within the normal range to start may have contributed to the cell uptake being lower than expected as the endoplasm of the cells may not be seeing a deficiency and not encouraging vesicle intake of D3. It may be that the additional D3 was not absorbed by the cells and was in fact transported to body fat for storage.

The test results respective of uptake were disappointing in that no significant delta was observed from the baseline to the two subsequent post test results. The test team's considered opinion was that as in the folate results, the pharmacokinetics of the test affected the results.

### Biomarker #4—Homocysteine

Observed measures—

Baseline—8.0 umol/L

45 min.—7.0 umol/L

75 min.—8.0 umol/L

This non-protein biomarker for measuring the impact of increasing B12 and folate is consistent with the vitamin B-12 increase and decrease. The level went down at the 45 min. interval post test and rose as to B12 dropped that the 75 min. interval.

It appeared that the rise and return to the baseline measure did indicate an increase in B levels that was significant enough to create a 1.0 umol/L change. This implies the uptake was significant for at least the B12 is aligned logically to this result.

Pre-test data showed a low White Blood Cell count at 3.7. The test baseline was at 4.8 and 5.4 at 45 and 5.2 at 1:15. Overall a significant increase during testing, however it appeared that some correction may have been made between the initial testing and subsequent testing. These results were confirmed with darkfield microscopy

Pre-test results for Red Blood Cells showed acceptable levels at 3.95. The test baseline was 4.07 and 4.2 at 45 and 4.18 at 1:15. Overall an increase that is not readily explainable other than the probable impact of the delivered structured water bioenergy affecting erythropoiesis and an increase in the production of red blood cells by accelerating the conversion of pluripotent stem cells in the intervals between draws.

The changes in Ig (immunoglobulin) markers A, E, G, and M were not significant between baseline and the subsequent post-test intervals. This supports the idea that while the WBC count, stimulated by the structured water, was up in total count that the Ig responses across all areas showed minimal changes and were within the normal ranges indicating an immune system improvement, not a problem. In viewing the white blood cells microscopically, the white blood cells showed a corresponding activity and increased motility that indicates a positive change from the baseline to the 45 min. result.

A 20 point glucose drop from baseline at 102 to 82 after at 45 minutes and further to 94 at 75 minutes A reasonable assumption was that a dramatic uptake of glucose occurred due to rapid metabolic changes induced by the bio-energy at the intracellular level.

O2 increases were consistently higher after testing (from 96 to 98) and from (98 to 99—unofficial post test) which showed an immediate as well as an overall improvement.

A urinalysis test was conducted via test strips and the results are summarized below.

Glucose: Negative all 3 results.

Bilirubin: this result showed a slightly raised above negative at the first elevated level referred to as “small” for all 3 tests. The pre-test was in mg/dl so no direct comparison by measure could be made.

Ketone: just above negative at trace 0.5 on day 1 test for all 3 tests. All above negative results may be attributed to low gluten diet or low carb diet which is typical in the lifestyle and more likely were attributed to the fasting. Ketones seen in the pre-test were reported as negative. This change implies that fat metabolizing was taking place as the cellular metabolic rate increased during the test, likely due to the bio-energy stimulating the reactions.

Specific gravity: Day one was 1.005 in all 3 tests, normal readings. The pre-test report shows specific gravity to be 1.010. All are within the normally expected tolerances.

Blood: Blood traces showed negative in all three tests, normal readings.

pH: Baseline to after 45 min showed a shift in pH from 6.0 to 6.5 where remained at 6.5 at 1:15, less acidic while still in an acceptable range. While a slight shift, the stability at 6.5 would have to be considered a positive measure, moving to a more neutral pH.

Protein: baseline showed trace with a measure index of 16, with the 45 min. measure remaining the same. However the 1:15 test showed a slight increase the trace plus at a measure index of 32. The rapid elevation of 16 mg/deciliter were it sustained might be an indication of a changed biochemical condition, related to impaired kidney function or a possible UTI, however no further measurements were taken nor suppositions able to be developed at this time.

Uro-bilinogen: baseline to 1:15 measurements all showed or negative it 0.2 mg/dL.

Nitrite: baseline showed a normal negative with a zero measure. At 45 min. and 1:15, the trace measured at 15. A normal read of this transition may imply presence of gram-negative bacteria however the CBC numbers for the WBC baseline and post 45 min. and 1:15 stayed within normal tolerances of between 4.8 and 5.2.

Leukocytes: all three measures showed negative readings at zero measure. Again referencing nitrite readings, the implication of gram-positive bacteria present does not align.

Effectively, the test's limited success adds another credible aspect of trans-dermal uses for structured water, at least water-soluble molecule delivery, in this case water soluble vitamin B12, without being affected by pharmacokinetic interaction.

### Example 9

### Subject 3

In accordance with methods described above, structured water, a humic enhancer and an organic phytoplankton concentrated supplement (5% of oral dosage) were delivered via a shower mechanism over a two minute span. The test protocol concentration was 5% delivery, or 1 mL of a 1 oz preparation which contains 10.5 g (10,500 mg) of Pure Whole Live Marine Phytoplankton which is a delivery comparable to what might be an oral supplement dosage, and conservative to avoid any complications with test subjects. No inorganic material or pharmaceuticals were applied. Test results were taken approximately 15 minutes before and 30 minutes after the subject showered.

The test subject for the tests given on two days was a 62 year old male volunteer in good health. A (12) hour fast was observed before each test.

Blood was drawn and the subjects red blood cells observed using darkfield microscopy. Changes in blood plasma and cells, visible in the local view screens from the digital camera attached to the dark field microscope (10,000×), showed instant changes including: white blood cells, which were at initially very low counts and lethargic, increased to healthy counts and became very motile (meaning active); red blood cell stress markers, which included cell wall deformity, Rouleau and clumping, completely disappeared; red blood cell physiology showed the majority of cells developed proper shape and appeared to be well hydrated and all red blood cells went from static to highly energized and active.

The microscopy clearly demonstrates that the administration of structured water was able to broadly change conditions in the entire blood supply, in a matter of minutes.

The subject was monitored for other physiological parameters:

Pulse Oximeter—Oxygen stayed the same at 98%

Blood Pressure—Supine systolic (laying flat resting) blood pressure was reduced by 4.5.

Salivary pH—pH improved from 5.5 to 6.0 showing a reduction in acidity or acidosis.

Blood glucose level dropped 15% showing increased high and immediate cellular activity which increased demand for available glucose.

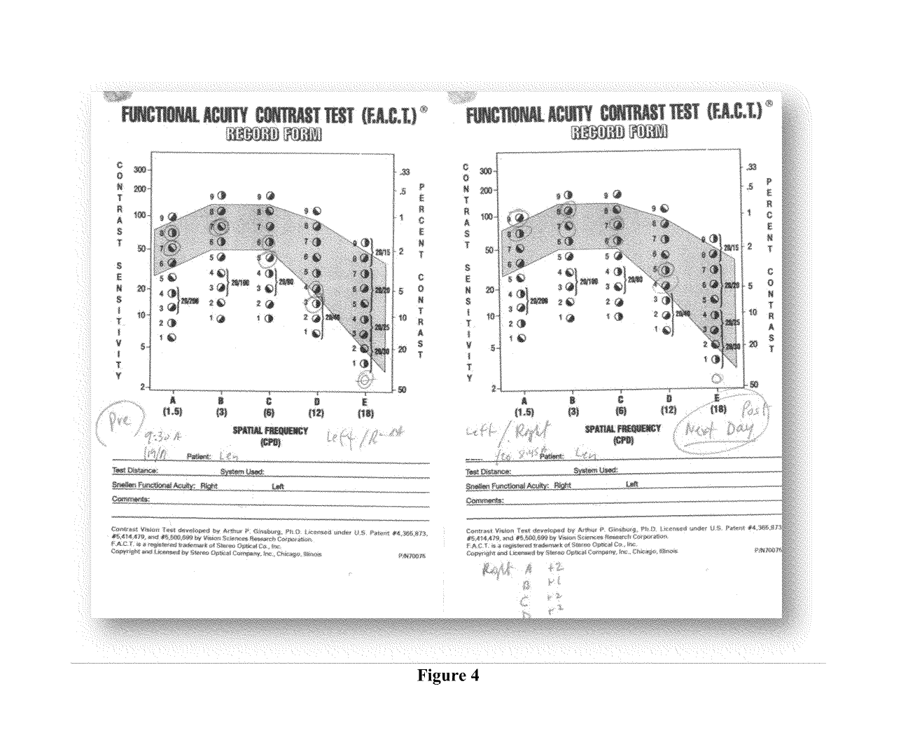

Visual Contrast Sensitivity test (a visual acuity test) showed a significant improvement in the body's toxic level across all (4) levels of the registered test levels; a process normally taking days to weeks. The results are shown in FIG. 4.

Bioelectrical Impedance—showed a 0.5 L shift in fluids from the intracellular to the extracellular location (plasma) indicating a change in osmosis, likely from a rapid release of toxins and fluids from the cells to the surrounding area due to higher metabolic activity.

Additional blood draws were taken four hours post testing and processed locally. Sodium, Chloride, CO2, Glucose and Alkaline Phosphates were shown to move from outside the normal range (low by 4-8%) at or within 1% of the acceptable ranges in all cases signifying the nutrition was delivered in a time frame and at a rate well above other mechanisms for delivery.

Immunoglobulin G was tested on the second day of testing and showed that, while the immune system had been significantly bolstered, it remained well within an acceptable normal range.

### Example 10

### Subject 4

A 49 year old male subject was tested with a protocol for determining the effectiveness of micronutrient delivery in a subject with ideal eating and exercise habits. The protocol was performed identically as it had with previous efficacy tests for Subject 2. Only a 5% phytoplankton solution was applied transdermally.

The subject listed a vertigo condition that he had been experiencing since a surgical procedure 3 years prior. The condition was likely a side-effect of anesthesia which often presents as central vertigo, versus peripheral vertigo, a condition attributed to physical issues of the inner ear. An immediate observation following the first of four days for the test was that the subject's vertigo intensity reduced significantly. Day 2 the subject self-tested using aVOR, Vestibular Reflex test, he used to determine if any improvement had occurred that was quantitative. Prior to the test, the number of repetitions of specific motions used was at 120 left and right, 200 up and down; after the test, 30 minutes, L&R improved to 170 and U&D to 229. Improvements continued. By the second test series a month later, the subject reported an 80% improvement from his qualitative/quantitative baseline. The overall result was near normal VOR physiological comfort in daily activities.

No biomarker assessment was made of unexpected result in the test. Deficiencies noted in Spectracell panels showed nothing prior that would have been significant in the noted changes. As the condition returns over several days post-test, the speculation is that the test process may have altered damaged neuropathways as a result of the structured water providing either a chelating effect or a free radical alteration at a neurological/optical junction. The reportable aspect of the two, one week test periods was that the structured water and delivered phytochemicals/micro nutrients positively affected the subject each time with near normalizing results for his central vertigo.

### Example 11

### Subjects 5 and 6

A test protocol was devised to test free radical changes before and after exposure to the structured water. A 21 year old and a 46 year old male were tested using only the structured water. In this test protocol, no surfactants were used or any micronutrients applied in order to remove any variables from the results. Both tests were run at 101 to 102 degrees F. shower with the durations of exposure being the same as used in previous tests with a two minute warm up period, a two minute period normally used for the application of surfactants and nutrients and a two minute rinse.; 6 minutes in total. The range in pH during delivery of the water was between 7.45 to 7.75 at +500 ORP.

To determine the results, each subject was tested before and after with an Oxidata urinalysis kit, a calorimetric reagent that serves as an easy method to determine urinary levels of malondialdehyde, an end product of lipid peroxidation. The test is a color charted test with a range of 0, minimal free radicals, to 5, a high level. The older subject showed no real difference in before and after with a 5 value in each case. Results however for the younger subject showed a drop from 5 to 3.5/3.75.

The test results imply that a change occurred in the younger however the degree of difficulty in color perception and in comparing test vials of urine samples to the provided charts. It was also determined that bathing may prompt better results with exposure exceeding 10 minutes, based on known transdermal uptake tests for water.

### Example 12

### All Test Subjects

In all test subjects reviewed over a 30 month period in testing involving nutrient molecule delivery, dark field microcopy was done for before and after review to ascertain changes in general CBC. The maximum observable magnification was 10,000×. The overwhelming majority of test subjects demonstrated various Rouleau conditions indicating that the conditions in the serum prior to testing. These conditions are normally attributed to acidosis or alkalosis or high levels of free radicals or other immune irritants. In the same majority the pre-test subject observations showed a variety of stress markers, low motility, viroids and bacteria common to many people, all based on immunological compromises created by food allergies, environmental toxicities etc. Post test these same subjects showed dramatic reductions in pathogens, increases in WBC counts (white blood cells), improved shape and hydration in RBC (red blood cells), elimination of RBC stress markers, reduction in lipid trails on the slides, and often dramatic changes in motility, typically in RBCs but frequently in WBCs. While limited scientific evidence of serum and cell physiology was recorded, other than specific registered in the protocols, tests done such as CLIA-10 urinary series showed corresponding improvements in urinary pH and specific gravity ranges that seemed to correlate in confirming the interstitial fluids, certainly the blood serum epigenetics had substantially improved in the 30-60 minute window of pre and post test results under the darkfield microcopy.

One of skill in the art will appreciate that substitutions and deviations from the above formulation may be permissible without departing from the spirit of the invention.

We claim:

1. A structured water composition having a cluster size between 4 and 5, a pH between about 6.5 and about 8.0, an ORP value of at least −300 and a resistance from about 1 to about 18 megaohms.

2. The structured water of claim 1 further comprising one or more agents selected from the group comprising dietary supplements, vitamins, phytochemicals, homeopathic agents and pharmacologically or biologically active agents.

3. The structured water of claim 2 further comprising an agent for enhancing transdermal delivery of an active agent wherein the enhancing agent is a surfactant or a humic acid derivative.

4. A method of manufacturing the structured water of claim 1 comprising:
a) filtering a water to at least 1 megaohm purity
b) passing the water through a media capable of producing a high negative ORP
c) modifying the pH of the water to be biologically safe

5. The method of claim 4 wherein the media is a tourmaline ceramic ball

6. The method of claim 4 wherein the water frequency is modified.

7. The method of claim 4 where in the pH is modified to about 6.5 and about 8.0 using a biologically acceptable acid.

8. The method of claim 4 wherein the acid is ascorbic acid.

9. A method of reducing oxidative stress in an organism comprising the administration of a structured water having a cluster size between 4 and 5 and an ORP value of at least −300, a pH between about 6.5 and about 8.0, and a resistance from about 1 to about 18 megaohms

10. The method of claim 9 where in the structured water is administered in combination with one or more agents selected from the group comprising dietary supplements, vitamins, phytochemicals, homeopathic agents and pharmacologically or biologically active agents.

11. The method of claim 10 wherein the structured water further comprises an agent for enhancing transdermal delivery of an active agent, wherein the enhancing agent is a surfactant or a humic acid derivative.

12. The method of claim 9 wherein the structured water is administered topically or internally.

13. The method of claim 9 wherein the structured water is administered to at least 80% of the external surface area of the organism to which it is applied.

14. A method of transdermally delivering an agent using the structured water of claim 1 comprising:
a) mixing an active agent into the structured water of claim; and
b) administering the structured water mixture to a organism in need thereof.

15. The method of claim 14 in which the one or more agents are selected from the group comprising dietary supplements, vitamins, phytochemicals, homeopathic agents and pharmacologically or biologically active agents.

16. The method of claim 14 wherein the composition further comprises an agent for enhancing transdermal delivery of an active agent, wherein the enhancing agent is a surfactant or a humic acid derivative.

17. The method of claim 14 wherein the structured water is administered to at least 80% of the external surface area of the organism to which it is applied.

18. A method of improving cellular energy comprising the administration of structured water according to claim 1 wherein the water has, a pH between about 6.5 and about 8.0 and a resistance from about 1 to about 18 megaohms and one or more agents selected from group comprising dietary supplements, vitamins, phytochemicals, homeopathic agents and pharmacologically or biologically active agents to an organism in need thereof.

19. The method of claim 18 wherein the composition further comprises an enhancing agent for enhancing transdermal delivery of an active agent, wherein the enhancing agent is a surfactant or a humic acid derivative.

20. A method of inducing folate production in an organism capable of producing folate comprising the administration of the structured water of claim 1 to an organism in need thereof.

21. A method of inducing folate production in an organism capable of producing folate comprising the administration of the structured water of claim 2 to an organism in need thereof.

22. A method of inducing folate production in an organism capable of producing folate comprising the administration of the structured water of claim 3 to an organism in need thereof.

23. A system for creating structured water comprising:
a) purifying water to at least 1 megohm purity
b) processing the water to achieve a water cluster size of equal to or less than 6 using infrared radiation; and
c) processing the water to achieve at negative ORP of at least −300.

24. The system of claim 23 wherein the water is purified using one or more of the following: particulate filters, absorbents, carbons, resins, reverse osmosis or distillation.

25. The system of claim 23 wherein the infrared radiation is supplied by tourmaline

26. The system of claim 23 wherein the cluster size is maintained by avoiding the use of sharp angles in plumbing the system.

27. The system of claim 23 wherein the pH is adjusted with ascorbic acid to a pH between 6 and 8.
